(19) United States
(12) Patent Application Publication
(10) Pub. No.: US 2022/0031603 A1
(43) Pub. Date: Feb. 3, 2022

(54) COMPOSITION FOR IMPROVING MICROFLORA CONTAINING HERBAL MEDICINE EXTRACT AS ACTIVE INGREDIENT

(71) Applicant: KBIOLAB CO., LTD., Daejeon (KR)

(72) Inventors: Tae Jong KIM, Daejeon (KR); Natpisit CHAITACHAWONG, Seoul (KR)

(73) Assignee: KBIOLAB CO., LTD., Daejeon (KR)

(21) Appl. No.: 17/414,153

(22) PCT Filed: Oct. 30, 2018
(86) PCT No.: PCT/KR2018/012993

§ 371 Date: Jun. 15, 2021

(30) Foreign Application Priority Data
Oct. 31, 2017 (KR) 10-2017-0144152
Oct. 31, 2017 (KR) 10-2017-0144153
Oct. 31, 2017 (KR) 10-2017-0144154
Oct. 31, 2017 (KR) 10-2017-0144155
Oct. 31, 2017 (KR) 10-2017-0144156
Oct. 31, 2017 (KR) 10-2017-0144157

(51) Int. Cl. A61K 8/9789; A61Q 17/00; A61Q 19/00; A61Q 5/02; A61Q 5/12

(52) U.S. Cl. CPC A61K 8/9789; A61Q 17/005; C12R 2001/45; A61Q 5/02; A61Q 5/12; A61Q 19/00

(57) ABSTRACT

Disclosed is a composition for improving microbial flora containing a herbal medicine extract as an active ingredient, wherein the composition can inhibit the growth of harmful bacteria and promote the growth of beneficial bacteria.

### TECHNICAL FIELD

The present disclosure relates to a composition for improving microbial flora, comprising a herbal medicine extract as an active ingredient.

### BACKGROUND ART

The human body is a complex and active ecosystem, and various microorganisms exist in the gastrointestine and skin of the human body. A microbial community existing in the gastrointestine and skin, etc., is called microbial flora, and 500 to 1000 or more different microorganisms coexist together to form the microbial flora. Intestinal microorganisms present in the gastrointestine synthesize substances such as vitamins and supply them to the human body, and play a role in suppressing infection by pathogenic microorganisms introduced through contaminated food.

Microorganisms on the skin affect the human physiologies such as human growth, nutrition, immunity, resistance to pathogenicity, etc., and play a role in blocking the invasion of external microorganisms, particularly pathogenic microorganisms. In addition, microorganisms residing on the skin are known to control the pH of the skin and perform a skin barrier function, and to affect a skin condition by changing the skin lipid content. For example, it is known that atopic patients have dry and cracked keratinized skin due to a decrease of the ceramide content in the stratum corneum, and Staphylococcus aureus inhabiting the skin is increasing extremely.

Since there are microorganisms that have a beneficial effect on the human body, it is necessary to promote the growth of beneficial bacteria while suppressing the growth of harmful bacteria, rather than simply removing (sterilizing) all microorganisms.

The present inventors completed the present disclosure as a result of conducting research for improving the microbial flora using natural products.

### DISCLOSURE

### Technical Problem

An object of the present disclosure is to provide a composition for improving microbial flora and a cosmetic composition for improving skin residential flora, comprising a herbal medicine extract as an active ingredient.

Another object of the present disclosure is to provide a composition for improving microbial flora and a cosmetic composition for improving skin residential flora, comprising a Persicae Semen extract as an active ingredient.

Still another object of the present disclosure is to provide a composition for improving microbial flora and a cosmetic composition for improving skin residential flora, comprising a Benincasa hispida Cogniaux (Benincasae Semen) extract as an active ingredient.

Yet another object of the present disclosure is to provide a composition for improving microbial flora and a cosmetic composition for improving skin residential flora, comprising a Dolichoris Semen extract as an active ingredient.

Yet another object of the present disclosure is to provide a composition for improving microbial flora and a cosmetic composition for improving skin residential flora, comprising a Mori Folium extract as an active ingredient.

Yet another object of the present disclosure is to provide a composition for improving microbial flora and a cosmetic composition for improving skin residential flora, comprising a Coicis Semen extract as an active ingredient.

Yet another object of the present disclosure is to provide a composition for improving microbial flora and a cosmetic composition for improving skin residential flora, comprising a Carthami Fructus extract as an active ingredient.

### Technical Solution

In an aspect, there is provided a composition for improving microbial flora, comprising a herbal medicine extract as an active ingredient.

In one embodiment of the present disclosure, the herbal medicine may be Persicae Semen, Benincasa hispida Cogniaux (Benincasae Semen), Dolichoris Semen, Mori Folium, Coicis Semen, or Carthami Fructus.

In one embodiment of the present disclosure, the composition for improving microbial flora, comprising a Persicae Semen extract as an active ingredient may be provided.

In one embodiment of the present disclosure, the composition for improving microbial flora, comprising a Benincasa hispida Cogniaux (Benincasae Semen) extract as an active ingredient may be provided.

In one embodiment of the present disclosure, the composition for improving microbial flora, comprising a Dolichoris Semen extract as an active ingredient may be provided.

In one embodiment of the present disclosure, the composition for improving microbial flora, comprising a Mori Folium extract as an active ingredient may be provided.

In one embodiment of the present disclosure, the composition for improving microbial flora, comprising a Coicis Semen extract as an active ingredient may be provided

In one embodiment of the present disclosure, the composition for improving microbial flora, comprising a Carthami Fructus extract as an active ingredient may be provided.

In one embodiment of the present disclosure, the herbal medicine extract may be extracted with one or more solvents selected from the group consisting of water, alcohol having 1 to 4 carbon atoms, ethyl acetate, diethyl ether, dichloromethane, and acetone.

In one embodiment of the present disclosure, the Persicae Semen extract may be extracted with one or more solvents selected from the group consisting of water, alcohol having 1 to 4 carbon atoms, ethyl acetate, diethyl ether, dichloromethane, and acetone.

In one embodiment of the present disclosure, the Benincasa hispida Cogniaux (Benincasae Semen) extract may be extracted with one or more solvents selected from the group consisting of water, alcohol having 1 to 4 carbon atoms, ethyl acetate, diethyl ether, dichloromethane, and acetone.

In one embodiment of the present disclosure, the Dolichoris Semen extract may be extracted with one or more solvents selected from the group consisting of water, alcohol having 1 to 4 carbon atoms, ethyl acetate, diethyl ether, dichloromethane, and acetone.

In one embodiment of the present disclosure, the Mori Folium extract may be extracted with one or more solvents selected from the group consisting of water, alcohol having 1 to 4 carbon atoms, ethyl acetate, diethyl ether, dichloromethane, and acetone.

In one embodiment of the present disclosure, the Coicis Semen extract may be extracted with one or more solvents selected from the group consisting of water, alcohol having 1 to 4 carbon atoms, ethyl acetate, diethyl ether, dichloromethane, and acetone.

In one embodiment of the present disclosure, the Carthami Fructus extract may be extracted with one or more solvents selected from the group consisting of water, alcohol having 1 to 4 carbon atoms, ethyl acetate, diethyl ether, dichloromethane, and acetone.

In one embodiment of the present disclosure, the herbal medicine extract may be included in an amount of 0.005 to 10% by weight based on the total weight of the composition.

In one embodiment of the present disclosure, the Persicae Semen extract may be included in an amount of 0.005 to 10% by weight based on the total weight of the composition.

In one embodiment of the present disclosure, the Benincasa hispida Cogniaux (Benincasae Semen) extract may be included in an amount of 0.005 to 10% by weight based on the total weight of the composition.

In one embodiment of the present disclosure, the Dolichoris Semen extract may be included in an amount of 0.005 to 10% by weight based on the total weight of the composition.

In one embodiment of the present disclosure, the Mori Folium extract may be included in an amount of 0.005 to 10% by weight based on the total weight of the composition.

In one embodiment of the present disclosure, the Coicis Semen extract may be included in an amount of 0.005 to 10% by weight based on the total weight of the composition.

In one embodiment of the present disclosure, the Carthami Fructus extract may be included in an amount of 0.005 to 10% by weight based on the total weight of the composition.

In one embodiment of the present disclosure, the composition may inhibit the growth of harmful bacteria and promote the growth of beneficial bacteria.

In one embodiment of the present disclosure, the harmful bacteria may be Staphylococcus aureus.

In one embodiment of the present disclosure, the beneficial bacteria may be Staphylococcus epidermidis.

In another aspect, there is provided a cosmetic composition for improving a skin residential flora, comprising a herbal medicine extract as an active ingredient.

In one embodiment of the present disclosure, the herbal medicine may be Persicae Semen, Benincasa hispida Cogniaux (Benincasae Semen), Dolichoris Semen, Mori Folium, Coicis Semen, or Carthami Fructus.

In one embodiment of the present disclosure, the cosmetic composition for improving a skin residential flora, comprising a Persicae Semen extract as an active ingredient may be provided.

In one embodiment of the present disclosure, the cosmetic composition for improving a skin residential flora, comprising a Benincasa hispida Cogniaux (Benincasae Semen) extract as an active ingredient may be provided.

In one embodiment of the present disclosure, the cosmetic composition for improving a skin residential flora, comprising a Dolichoris Semen extract as an active ingredient may be provided.

In one embodiment of the present disclosure, the cosmetic composition for improving a skin residential flora, comprising a Mori Folium extract as an active ingredient may be provided.

In one embodiment of the present disclosure, the cosmetic composition for improving a skin residential flora, comprising a Coicis Semen extract as an active ingredient may be provided.

In one embodiment of the present disclosure, the cosmetic composition for improving a skin residential flora, comprising a Carthami Fructus extract as an active ingredient may be provided.

In one embodiment of the present disclosure, the cosmetic composition may be a formulation selected from the group consisting of gels, emulsions, suspensions, microemulsions, microcapsules, microgranules, ionic vesicle dispersants, non-ionic vesicle dispersants, creams, skin toners, lotions, powders, ointments, sprays, concealer sticks, aerosols, and mask packs.

In another aspect, there is provided a detergent composition, comprising a herbal medicine extract as an active ingredient.

In one embodiment of the present disclosure, the herbal medicine may be Persicae Semen, Benincasa hispida Cogniaux (Benincasae Semen), Dolichoris Semen, Mori folium, Coicis Semen, or Carthami Fructus.

In one embodiment of the present disclosure, the detergent composition, comprising a Persicae Semen extract as an active ingredient may be provided.

In one embodiment of the present disclosure, the detergent composition, comprising a Benincasa hispida Cogniaux (Benincasae Semen) extract as an active ingredient may be provided.

In one embodiment of the present disclosure, the detergent composition, comprising a Dolichoris Semen extract as an active ingredient may be provided.

In one embodiment of the present disclosure, the detergent composition, comprising a Mori folium extract as an active ingredient may be provided.

In one embodiment of the present disclosure, the detergent composition, comprising a Coicis Semen extract as an active ingredient may be provided.

In one embodiment of the present disclosure, the detergent composition, comprising a Carthami Fructus extract as an active ingredient may be provided.

In one embodiment of the present disclosure, the detergent may be selected from the group consisting of shampoos, rinses, hair conditioners, body washes, soaps, hand washes, laundry detergents, and kitchen detergents.

### Advantageous Effects

A Persicae Semen extract, a Benincasa hispida Cogniaux (Benincasae Semen) extract, a Dolichoris Semen extract, a Mori folium extract, a Coicis Semen extract, or a Carthami Fructus extract may be usefully used for a use of improving microbial flora and skin residential flora, because they may promote growth of the beneficial bacteria, while inhibiting growth of harmful bacteria.

### BRIEF DESCRIPTION OF THE DRAWINGS

FIG. 1 shows the results of measuring the change in a growth rate of Staphylococcus epidermidis and Staphylococcus aureus independently by the treatment of a Persicae Semen extract.

FIG. 2 shows a growth change by the treatment of the Persicae Semen extract extracted with methanol in the case of a mixed culture of Staphylococcus epidermidis and Staphylococcus aureus.

FIG. 3 shows a growth change by the treatment of the Persicae Semen extract extracted with 95% ethanol in the case of a mixed culture of Staphylococcus aureus and

Staphylococcus epidermidis.

FIG. 4 shows a growth change according to a treatment concentration of the Persicae Semen extract extracted with 95% ethanol in the case of a mixed culture of Staphylococcus aureus and Staphylococcus epidermidis.

FIG. 5 shows the results of measuring the change in a growth rate of Staphylococcus epidermidis and Staphylococcus aureus independently by the treatment of a Benincasa hispida Cogniaux (Benincasae Semen) extract.

FIG. 6 shows a growth change by the treatment of the Benincasa hispida Cogniaux (Benincasae Semen) extract extracted with methanol in the case of a mixed culture of Staphylococcus epidermidis and Staphylococcus aureus.

FIG. 7 shows a growth change by the treatment of the Benincasa hispida Cogniaux (Benincasae Semen) extract extracted with 95% ethanol in the case of a mixed culture of Staphylococcus aureus and Staphylococcus epidermidis.

FIG. 8 shows a growth change according to the treatment concentration of the Benincasa hispida Cogniaux (Benincasae Semen) extract extracted with 95% ethanol in the case of a mixed culture of Staphylococcus aureus and Staphylococcus epidermidis.

FIG. 9 shows the results of measuring the change in a growth rate of Staphylococcus epidermidis and Staphylococcus aureus independently by the treatment of a Dolichoris Semen extract.

FIG. 10 shows a growth change by the treatment of the Dolichoris Semen extract extracted with methanol in the case of a mixed culture of Staphylococcus epidermidis and Staphylococcus aureus.

FIG. 11 shows a growth change by the treatment of the Dolichoris Semen extract extracted with 95% ethanol in the case of a mixed culture of Staphylococcus aureus and Staphylococcus epidermidis.

FIG. 12 shows a growth change according to the treatment concentration of the Dolichoris Semen extract extracted with 95% ethanol in the case of a mixed culture of Staphylococcus aureus and Staphylococcus epidermidis.

FIG. 13 shows the results of measuring the change in a growth rate of Staphylococcus epidermidis and Staphylococcus aureus independently by the treatment of a Mori Folium extract.

FIG. 14 shows a growth change by the treatment of the Mori Folium extract extracted with methanol in the case of a mixed culture of Staphylococcus epidermidis and Staphylococcus aureus.

FIG. 15 shows a growth change by the treatment of the Mori Folium extract extracted with 95% ethanol in the case of a mixed culture of Staphylococcus aureus and Staphylococcus epidermidis.

FIG. 16 shows a growth change according to the treatment concentration of the Mori Folium extract extracted with 95% ethanol in the case of a mixed culture of Staphylococcus aureus and Staphylococcus epidermidis.

FIG. 17 shows the results of measuring the change in a growth rate of Staphylococcus epidermidis and Staphylococcus aureus independently by the treatment of a Coicis Semen extract.

FIG. 18 shows a growth change by the treatment of the Coicis Semen extract extracted with methanol in the case of a mixed culture of Staphylococcus epidermidis and Staphylococcus aureus.

FIG. 19 shows a growth change by the treatment of the Coicis Semen extract extracted with 95% ethanol in the case of a mixed culture of Staphylococcus aureus and Staphylococcus epidermidis.

FIG. 20 shows a growth change according to the treatment concentration of the Coicis Semen extract extracted with 95% ethanol in the case of a mixed culture of Staphylococcus aureus and Staphylococcus epidermidis.

FIG. 21 shows the results of measuring the change in a growth rate of Staphylococcus epidermidis and Staphylococcus aureus independently by the treatment of a Carthami Fructus extract.

FIG. 22 shows a growth change by the treatment of the Carthami Fructus extract extracted with methanol in the case of a mixed culture of Staphylococcus epidermidis and Staphylococcus aureus.

FIG. 23 shows a growth change by the treatment of the Carthami Fructus extract extracted with 95% ethanol in the case of a mixed culture of Staphylococcus aureus and Staphylococcus epidermidis.

### BEST MODE

In order to achieve the object, one aspect of the present disclosure provides a composition for improving microbial flora, comprising a herbal medicine extract as an active ingredient.

In one embodiment of the present disclosure, the herbal medicine may be Persicae Semen, Benincasa hispida Cogniaux (Benincasae Semen), Dolichoris Semen, Mori Folium, Coicis Semen, or Carthami Fructus.

In one embodiment of the present disclosure, the composition for improving microbial flora, comprising a Persicae Semen extract as an active ingredient may be provided.

In one embodiment of the present disclosure, the composition for improving microbial flora, comprising a Benincasa hispida Cogniaux (Benincasae Semen) extract as an active ingredient may be provided.

In one embodiment of the present disclosure, the composition for improving microbial flora, comprising a Dolichoris Semen extract as an active ingredient may be provided.

In one embodiment of the present disclosure, the composition for improving microbial flora, comprising a Mori Folium extract as an active ingredient may be provided.

In one embodiment of the present disclosure, the composition for improving microbial flora, comprising a Coicis Semen extract as an active ingredient may be provided.

In one embodiment of the present disclosure, the composition for improving microbial flora, comprising a Carthami Fructus extract as an active ingredient may be provided.

As used herein, the term ‘microbial flora’ refers to a microbial community including all of bacteria, fungi, and other prokaryotes existing in the human body, and mainly inhabits the gastrointestine and skin. For example, beneficial intestinal bacteria inhabiting the gastrointestine include Lactobacillus and Bifidobacterium, and as harmful intestinal bacteria, Clostridium is known.

As used herein, the term ‘improvement’ refers to a state in which the constitution of the microbial flora is changed to be beneficial to the human body due to the administration of the composition.

Persicae Semen, which is used as an active ingredient in the composition for improving the microbial flora of the present disclosure, refers to a drug made from the seeds of Prunus persica or Prunus davidiana belonging to the Rosaceae family, and is known as representing a pharmacological action such as vasodilator action, anti-inflammatory action, anti-allergic action, etc. However, there is no known information about the efficacy of improving the microbial flora.

Benincasa hispida Cogniaux (Benincasae Semen) used as an active ingredient in the composition for improving the microbial flora according to the present disclosure refers to the mature seeds of wax gourd (Benincasa hispida), an annual creeper belonging to the Cucurbitaceae family. The Benincasa hispida Cogniaux (Benincasae Semen) contains saponin, linoleic acid, oleic acid, and unsaturated fatty acids, etc., and has been used for expectorant, antipyretic, diuretic, anti-inflammatory, skin care, etc., from the past. In addition, it is known to be effective in preventing gastric ulcer, inhibiting histamine secretion, representing anti-tumor, and anti-obesity, and preventing hypertension, but there is no known information about the efficacy of improving the microbial flora.

Dolichoris Semen, which is used as an active ingredient in the composition for improving the microbial flora according to the present disclosure, refers to the seeds of Dolichos lablab Linne belonging to the Fabaceae family, and has been used for diarrhea, lobster, vomiting, and indigestion due to chronic diseases from the past. In addition, anti-cancer action and immune-increasing action are known, but there is no known information about the efficacy of improving the microbial flora.

Mori Folium, which is used as an active ingredient of the composition for improving the microbial flora of the present disclosure, refers to dried leaves of Morus alba Linne or Morus bombycis Koidz, which are members of the Moraceae family. It is known that it is effective in dieting by inhibiting the increase of triglycerides in the body and has whitening activity, but there is no known information about the efficacy of improving the microbial flora.

Coicis Semen, which is used as an active ingredient of the composition for improving the microbial flora of the present disclosure, refers to a seed from which the seed coat of Coix lacryma-jobi Linne var. ma-yuen Stapf is removed. It has been used for edema, diarrhea, anorexia, antipyretic action, and drainage from the past, and is known as representing pharmacological action such as anti-cancer, skeletal muscle contraction, sedation, analgesic and antipyretic action. However, there is no known information about the efficacy of improving the microbial flora.

Carthami Fructus used as an active ingredient in the composition for improving the microbial flora of the present disclosure refers to the fruit of Carthamus tinctorius (safflower), and contains a large amount of vitamin E to prevent aging and enhance reproductive function. In addition, since it contains a lot of linoleic acid, it is known to be effective in the prevention and treatment of arteriosclerosis caused by excessive cholesterol, but there is no known information about the efficacy of improving the microbial flora.

In one embodiment of the present disclosure, the Persicae Semen extract may be prepared according to a conventional method known in the art. For example, after pulverizing the Persicae Semen, a solvent commonly used for extraction may be added, and extraction may be performed at an appropriate temperature and pressure to prepare the Persicae Semen extract.

In one embodiment of the present disclosure, the Benincasa hispida Cogniaux (Benincasae Semen) extract may be prepared according to a conventional method known in the art. For example, after pulverizing the Benincasa hispida Cogniaux (Benincasae Semen), a solvent commonly used for extraction may be added, and extraction may be performed at an appropriate temperature and pressure to prepare the Benincasa hispida Cogniaux (Benincasae Semen) extract.

In one embodiment of the present disclosure, the Dolichoris Semen extract may be prepared according to a conventional method known in the art. For example, after pulverizing the Dolichoris Semen, a solvent commonly used for extraction may be added, and extraction may be performed at an appropriate temperature and pressure to prepare the Dolichoris Semen extract.

In one embodiment of the present disclosure, the Mori Folium extract may be prepared according to a conventional method known in the art. For example, after pulverizing the Mori Folium, a solvent commonly used for extraction may be added, and extraction may be performed at an appropriate temperature and pressure to prepare the Mori Folium extract.

In one embodiment of the present disclosure, the Coicis Semen extract may be prepared according to a conventional method known in the art. For example, after pulverizing the Coicis Semen, a solvent commonly used for extraction may be added, and extraction may be performed at an appropriate temperature and pressure to prepare the Coicis Semen extract.

In one embodiment of the present disclosure, the Carthami Fructus extract may be prepared according to a conventional method known in the art. For example, after pulverizing the Carthami Fructus, a solvent commonly used for extraction may be added, and extraction may be performed at an appropriate temperature and pressure to prepare the Carthami Fructus extract.

In one embodiment of the present disclosure, the herbal medicine extract may be extracted with one or more solvents selected from the group consisting of water, alcohol having 1 to 4 carbon atoms, ethyl acetate, diethyl ether, dichloromethane, and acetone.

In one embodiment of the present disclosure, the Persicae Semen extract may be extracted with one or more solvents selected from the group consisting of water, alcohol having 1 to 4 carbon atoms, ethyl acetate, diethyl ether, dichloromethane, and acetone.

In one embodiment of the present disclosure, the Benincasa hispida Cogniaux (Benincasae Semen) extract may be extracted with one or more solvents selected from the group consisting of water, alcohol having 1 to 4 carbon atoms, ethyl acetate, diethyl ether, dichloromethane, and acetone.

In one embodiment of the present disclosure, the Dolichoris Semen extract may be extracted with one or more solvents selected from the group consisting of water, alcohol having 1 to 4 carbon atoms, ethyl acetate, diethyl ether, dichloromethane, and acetone.

In one embodiment of the present disclosure, the Mori Folium extract may be extracted with one or more solvents selected from the group consisting of water, alcohol having 1 to 4 carbon atoms, ethyl acetate, diethyl ether, dichloromethane, and acetone.

In one embodiment of the present disclosure, the Coicis Semen extract may be extracted with one or more solvents selected from the group consisting of water, alcohol having 1 to 4 carbon atoms, ethyl acetate, diethyl ether, dichloromethane, and acetone.

In one embodiment of the present disclosure, the Carthami Fructus extract may be extracted with one or more solvents selected from the group consisting of water, alcohol having 1 to 4 carbon atoms, ethyl acetate, diethyl ether, dichloromethane, and acetone.

Meanwhile, the herbal medicine extract, the Persicae Semen extract, the Benincasa hispida Cogniaux (Benincasae Semen) extract, the Dolichoris Semen extract, the Mori Folium extract, the Coicis Semen extract, or the Carthami Fructus extract may include both the extract by solvents as disclosed above, and the extract that has undergone a conventional purification process. For example, fractions obtained through various purification methods, which are additionally carried out, such as separation using an ultrafiltration membrane having a constant molecular weight cut-off value, separation by various chromatography (prepared for separation according to size, charge, hydrophobicity, or affinity), etc. may also be included in the extract of the present disclosure. In addition, the herbal medicine extracts, the Persicae Semen extract, the Benincasa hispida Cogniaux (Benincasae Semen) extract, the Dolichoris Semen extract, the Mori Folium extract, the Coicis Semen extract, or the Carthami Fructus extract may be prepared in a powdered form by the additional process such as reduced pressure distillation and freeze drying or spray drying.

In one embodiment of the present disclosure, the composition for improving the microbial flora may contain 0.005 to 10% by weight, or 1 to 5% by weight of the herbal medicine extract based on the total weight of the composition, and a content of the active ingredient may be appropriately adjusted according to the method and purpose of use of the composition.

In one embodiment of the present disclosure, the composition for improving the microbial flora may contain 0.005 to 10% by weight, or 1 to 5% by weight of the Persicae Semen extract based on the total weight of the composition, and content of the active ingredient may be appropriately adjusted according to the method and purpose of use of the composition.

In one embodiment of the present disclosure, the composition for improving the microbial flora may contain 0.005 to 10% by weight, or 1 to 5% by weight of the Benincasa hispida Cogniaux (Benincasae Semen) extract based on the total weight of the composition, and content of the active ingredient may be appropriately adjusted according to the method and purpose of use of the composition.

In one embodiment of the present disclosure, the composition for improving the microbial flora may contain 0.005 to 10% by weight, or 1 to 5% by weight of the Dolichoris Semen extract based on the total weight of the composition, and content of the active ingredient may be appropriately adjusted according to the method and purpose of use of the composition.

In one embodiment of the present disclosure, the composition for improving the microbial flora may contain 0.005 to 10% by weight, or 1 to 5% by weight of the Mori Folium extract based on the total weight of the composition, and content of the active ingredient may be appropriately adjusted according to the method and purpose of use of the composition.

In one embodiment of the present disclosure, the composition for improving the microbial flora may contain 0.005 to 10% by weight, or 1 to 5% by weight of the Coicis Semen extract based on the total weight of the composition, and content of the active ingredient may be appropriately adjusted according to the method and purpose of use of the composition.

In one embodiment of the present disclosure, the composition for improving the microbial flora may contain 0.005 to 10% by weight, or 1 to 5% by weight of the Carthami Fructus extract based on the total weight of the composition, and content of the active ingredient may be appropriately adjusted according to the method and purpose of use of the composition.

In one embodiment of the present disclosure, the composition for improving the microbial flora may promote the growth of beneficial bacteria while inhibiting the growth of harmful bacteria. When the proportion of beneficial bacteria in the microbial flora increases, infection by pathogenic microorganisms may be prevented, immunity may be improved, a skin condition may be improved, and body odor may be improved.

In one embodiment of the present disclosure, the harmful bacteria may be one or more selected from the group consisting of Propionibacterium, Staphylococcus, Streptococcus, Candida, Acinetobacter, Corynebacterium, and Pseudomonas, but are not limited thereto. In addition, the harmful bacteria may be Propionibacterium acnes or Candida albicans.

In one embodiment of the present disclosure, the harmful bacteria may be Staphylococcus aureus. Staphylococcus aureus is a Gram-positive facultative anaerobic bacterium that is commonly present on the skin and nasal surfaces of healthy people or livestock. S. aureus is known as a causative agent of purulent diseases such as skin suppuration, otitis media and cystitis as well as food poisoning.

In one embodiment of the present disclosure, the beneficial bacteria may be Staphylococcus epidermidis (S. epidermidis). S. epidermidis is a Gram-positive bacterium and is generally present on the skin and mucous membranes of humans or livestock. It is known that S. epidermidis produces glycerin to sustain the moisturizing effect of the skin (J. Invest. Dermatol., 1960, 34:171-174), and synthesizes organic acids to keep the skin acidic (Br. J. Dermatol. 1986, 80:279-281), and produces antibiotics to inhibit the skin growth of harmful microorganisms such as S. aureus (N. Engl. J. Med., 1998, 339:520-532; Nature, 2010, 465:346-349; Peptides, 2010, 31:1661-1668; J. Invest. Dermatol., 2010, 130:192-200), which has a beneficial effect on the skin.

Another aspect of the present disclosure provides a cosmetic composition for improving a skin residential flora, containing a herbal medicine extract as an active ingredient.

In one embodiment of the present disclosure, the herbal medicine may be Persicae Semen, Benincasa hispida Cogniaux (Benincasae Semen), Dolichoris Semen, Mori Folium, Coicis Semen, or Carthami Fructus.

In one embodiment of the present disclosure, the cosmetic composition for improving a skin residential flora, comprising a Persicae Semen extract as an active ingredient may be provided.

In one embodiment of the present disclosure, the cosmetic composition for improving a skin residential flora, comprising a Benincasa hispida Cogniaux (Benincasae Semen) extract as an active ingredient may be provided.

In one embodiment of the present disclosure, the cosmetic composition for improving a skin residential flora, comprising a Dolichoris Semen extract as an active ingredient may be provided.

In one embodiment of the present disclosure, the cosmetic composition for improving a skin residential flora, comprising a Mori Folium extract as an active ingredient may be provided.

In one embodiment of the present disclosure, the cosmetic composition for improving a skin residential flora, comprising a Coicis Semen extract as an active ingredient may be provided.

In one embodiment of the present disclosure, the cosmetic composition for improving a skin residential flora, comprising a Carthami Fructus extract as an active ingredient may be provided.

As used herein, the term ‘skin residential flora’ refers to a microbial community existing on the skin, and mainly inhabits the skin surface such as the scalp and pores. The skin residential flora includes beneficial skin bacteria that have beneficial effects on the skin, such as maintaining skin moisture and removing active oxygen, and harmful skin bacteria that cause skin troubles such as acne and suppuration.

In one embodiment of the present disclosure, the cosmetic composition may be a formulation selected from the group consisting of gels, emulsions, suspensions, microemulsions, microcapsules, microgranules, ionic vesicle dispersants, non-ionic vesicle dispersants, creams, skin toners, lotions, powders, ointments, sprays, concealer sticks, aerosols, and mask packs. Examples of the cosmetic composition may include basic cosmetics, makeup cosmetics, hair cosmetics, body cosmetics, etc., and may be appropriately selected according to the purpose.

For example, the cosmetic composition may be formulated as solutions, suspensions, emulsions, pastes, gels, creams, lotions, powders, soaps, surfactant-containing cleansing, oils, powder foundations, emulsion foundations, wax foundations, and sprays, but is not limited thereto. More specifically, it may be formulated as basic cosmetics such as softening lotions, nourishing lotions, lotions, body lotions, nourishing creams, massage creams, moisture creams, hand creams, essences, eye creams, cleansing creams, cleansing foams, cleansing waters, packs, gels, patches, oil-in-water (O/W) type or water-in-oil (W/O) type, color cosmetics such as lipsticks, makeup bases, or foundations, cleanings such as toothpaste or mouthwash, hair fixatives such as hair tonic, gel, mousse, etc., and cosmetic compositions for hair such as hair tonics or hair dyes.

In one embodiment of the present disclosure, when the formulation of the cosmetic composition is a solution or emulsion, a solvent, solubilizer or emulsifier is used as a carrier component, and examples thereof include water, ethanol, isopropanol, ethyl carbonate, ethyl acetate, benzyl alcohol, benzyl benzoate, propylene glycol, 1,3-butylglycol oil, glycerol fatty esters, fatty acid esters of polyethylene glycol or sorbitan.

In one embodiment of the present disclosure, when the formulation of the cosmetic composition is a suspension, as a carrier component, a liquid diluent such as water, ethanol or propylene glycol, ethoxylated isostearyl alcohol, suspending agent such as polyoxyethylene sorbitol ester, and polyoxyethylene sorbitan esters, microcrystalline cellulose, aluminum metahydroxide, bentonite, agar or tracanth, etc., may be used.

In one embodiment of the present disclosure, when the formulation of the cosmetic composition is pastes, creams or gels, as a carrier component, animal oil, vegetable oil, wax, paraffin, starch, tragacanth, cellulose derivative, polyethylene glycol, silicone, bentonite, silica, talc or zinc oxide may be used.

In one embodiment of the present disclosure, when the formulation of the cosmetic composition is a powder or a spray, as a carrier component, lactose, talc, silica, aluminum hydroxide, calcium silicate, or polyamide powder may be used, and especially in the case of a spray, may additionally contain propellants such as chlorofluorohydrocarbons, propane/butane or dimethyl ether.

In addition, in the cosmetic composition of each formulation, other ingredients other than the herbal medicine extract, the Persicae Semen extract, the Benincasa hispida Cogniaux (Benincasae Semen) extract, the Dolichoris Semen extract, the Mori Folium extract, the Coicis Semen extract or the Carthami Fructus extract may be optionally selected and blended according to the formulation or purpose of use of the cosmetic composition. In addition, the composition of each formulation may contain various bases and additives necessary for formulation of the formulation, and within the range that does not reduce the effect, may contain known compounds of nonionic surfactants, silicone polymers, extender pigments, fragrances, antiseptic substances, disinfectants, oxidative stabilizers, organic solvents, ionic or nonionic thickeners, emollients, antioxidants, free radical scavengers, opacifiers, stabilizers, emollients, silicones, α-hydroxy acids, antifoaming agents, humectants, vitamins, preservatives, surfactants, fillers, polymers, propellants, basifying agents, acidifying agents, or coloring agents, etc.

Another aspect of the present disclosure provides a detergent composition, comprising a herbal medicine extract as an active ingredient.

In one embodiment of the present disclosure, the herbal medicine may be Persicae Semen, Benincasa hispida Cogniaux (Benincasae Semen), Dolichoris Semen, Mori Folium, Coicis Semen, or Carthami Fructus.

In one embodiment of the present disclosure, the detergent composition comprising a Persicae Semen extract as an active ingredient may be provided.

In one embodiment of the present disclosure, the detergent composition comprising a Benincasa hispida Cogniaux (Benincasae Semen) extract as an active ingredient may be provided.

In one embodiment of the present disclosure, the detergent composition comprising a Dolichoris Semen extract as an active ingredient may be provided.

In one embodiment of the present disclosure, the detergent composition comprising a Mori Folium extract as an active ingredient may be provided.

In one embodiment of the present disclosure, the detergent composition comprising a Coicis Semen extract as an active ingredient may be provided.

In one embodiment of the present disclosure, the detergent composition comprising a Carthami Fructus extract as an active ingredient may be provided.

In one embodiment of the present disclosure, the detergent composition may be selected from the group consisting of shampoos, rinses, hair conditioners, body washes, soaps, hand washes, laundry detergents, and kitchen detergents. In addition, various bases and additives other than the herbal medicine extract, Persicae Semen extract, Benincasa hispida Cogniaux (Benincasae Semen) extract, Dolichoris Semen extract, Mori Folium extract, Coicis Semen extract, or Carthami Fructus extract may be included according to the purpose of use.

### DETAILED DESCRIPTION OF EMBODIMENTS

Hereinafter, one or more specific embodiments will be described in more detail through examples. However, these examples are for illustrative purposes only and the scope of the present disclosure is not limited to these examples.

### Example 1: Preparation of Persicae Semen Extracts

To 5 g of Persicae Semen, 50 ml of 95% ethanol or methanol (medicament:solvent ratio of 1:10) was added, and the primary extract was obtained by shaking in a constant temperature water bath at 50° C. for 3 hours. The primary extract was filtered with a filter paper, and the filtered extract was concentrated and dried to prepare the Persicae Semen extract. 50 mg of the dried Persicae Semen extract was dissolved in 1 ml of each extraction solvent, and solids insoluble in the solvent were removed by centrifugation, and only the supernatant was used as a sample.

### Example 2: Changes in Growth of Microorganisms by Persicae Semen Extract

2-1. Microorganisms and Culture Media

Staphylococcus aureus (Hereinafter referred to as S. aureus) was used as the skin harmful bacteria, and Staphylococcus epidermidis (Hereinafter referred to as S. epidermidis) was used as the skin beneficial bacteria.

S. aureus KCTC 1916 and S. epidermidis KCTC 1917 which were used in the experiment, were purchased from the Center for Biological Resources (Korean Collection for Type Cultures, Korea). The composition of the medium used for culturing the two microorganisms is shown in Table 1 below.

TABLE 1

| | | | |
|---|---|---|---|
| S. epidermidis | NB medium | Nutrient Broth | 0.8 g |
| KCTC 1917 | (100 ml) | (Agar) | 1.5 g |
| | | Water | Fill to a total |
| | | | volume of 100 ml |
| S. aureus | TSB medium | Tryptic Soy Broth | 3.0 g |
| KCTC 1916 | (100 ml) | (Agar) | 1.5 g |
| | | Water | Fill to a total |
| | | | volume of 100 ml |

2-2. Changes in Growth of Individual Skin Resident Microorganisms by Persicae Semen Extract

S. aureus KCTC 1916 strain stock was spread on TSB (Tryptic Soy Broth, Becton Dickinson and Company, USA) agar plate and cultured at 37° C. for 24 hours. Then, one single colony was picked up and inoculated into 5 ml of TSB liquid medium, and pre-cultured overnight at 37° C. and 250 rpm. The pre-cultured S. aureus KCTC 1916 strain was inoculated into 20 ml of TSB liquid medium so that O.D.₆₀₀ became 0.05, and 200 μl (1% (v/v)) of Persicae Semen extract extracted with methanol was added. In the control group, methanol was added at a concentration of 1% (v/v) instead of the Persicae Semen extract. Then, the growth degree was confirmed by measuring the absorbance at 600 nm using an Optizen 2120 UV plus spectrophotometer (Mecasys Co., Ltd., Daejeon, Korea) every 1 hour while culturing at 37° C. and 250 rpm.

Also, a stock of S. epidermidis KCTC 1917 was cultured in the same manner as above to measure the growth rate, except that the stock was cultured by spreading on an NB (nutrients broth, Becton Dickinson and Company) agar plate.

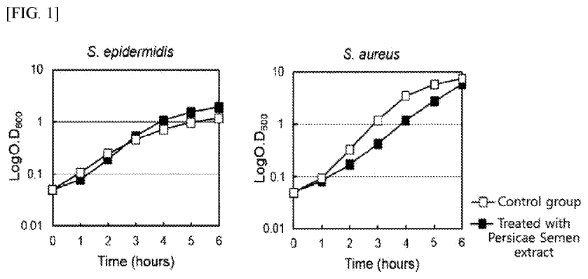

As a result of the measurement, as shown in FIG. 1, it could be confirmed that by the Persicae Semen extract extracted with methanol, the growth rate of S. epidermidis was increased and the growth rate of S. aureus was decreased. Specifically, the doubling time of S. epidermidis was decreased by about 38.42% in the group treated with the Persicae Semen extract (47.55 minutes) compared to the control group (77.22 minutes). On the other hand, the doubling time of S. aureus was increased by about 32.49% in the group treated with the Persicae Semen extract (46.53 minutes) compared to the control group (35.12 minutes).

2-3. Changes in Growth of Skin Resident Microorganisms by Persicae Semen Extract Extracted with Methanol

Microorganisms were pre-cultured in the same manner as in Example 2-2, and the pre-cultured S. aureus KCTC 1916 strain and S. epidermidis KCTC 1917 strain were mixed and inoculated in 20 ml of NB liquid medium so that O.D.₆₀₀ became 0.05. 200 μl (1% (v/v)) of Persicae Semen extract extracted with methanol was added, and in the case of control group, methanol was added at a concentration of 1% (v/v), followed by incubation at 37° C. and 250 rpm for 3 hours. Thereafter, 100 μl of the culture solution was spread on the NB agar plate, and cultured at 37° C. for 24 hours to confirm the colony forming unit (CFU).

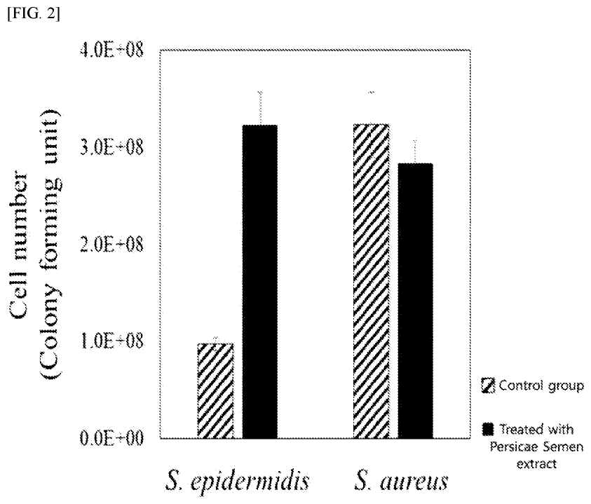

As a result, as shown in FIG. 2, it could be confirmed that by the treatment of the Persicae Semen extract extracted with methanol, the growth of S. epidermidis was remarkably increased and the growth of S. aureus was somewhat decreased.

2-4. Changes in Growth of Skin Resident Microorganisms by Persicae Semen Extract Extracted with 95% Ethanol

Except for the treatment using the Persicae Semen extract extracted with 95% ethanol at different concentrations (0 to 0.5 g/l), the growth degree of microorganisms was confirmed in the same manner as in 2-3 above.

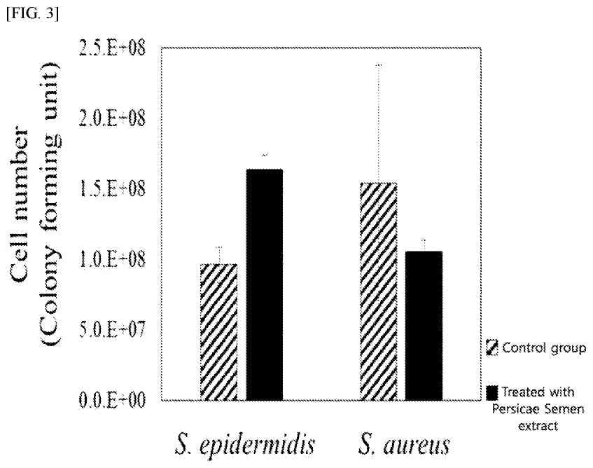

As a result, as shown in FIG. 3, it could be confirmed that by the treatment of the Persicae Semen extract extracted with 95% ethanol, the growth of S. aureus was remarkably decreased and the growth of S. epidermidis was increased.

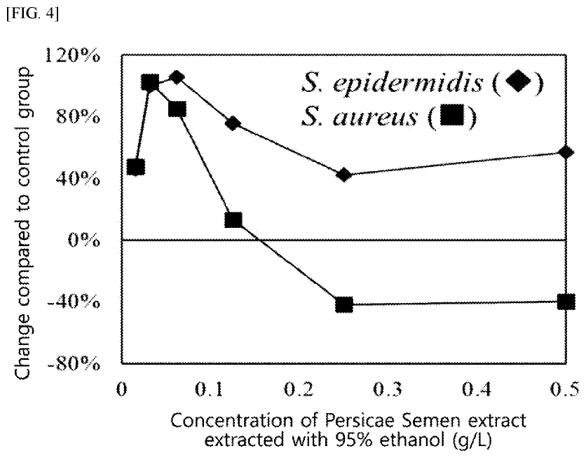

In addition, the degree of microbial growth was confirmed by the treatment of the Persicae Semen extract extracted with 95% ethanol at different concentrations (0 to 0.5 g/l). As a result, as shown in FIG. 4, it could be confirmed that by the treatment of the Persicae Semen extract extracted with 95% ethanol, the growth of S. epidermidis was increased and the growth of S. aureus was remarkably decreased.

### Example 3: Preparation of Benincasa hispida Cogniaux (Benincasae Semen) Extract

To 5 g of Benincasa hispida Cogniaux (Benincasae Semen), 50 ml of 95% ethanol or methanol (medicament:solvent ratio of 1:10) was added, and the primary extract was obtained by shaking in a constant temperature water bath at 50° C. for 3 hours. The primary extract was filtered with a filter paper, and the filtered extract was concentrated and dried to prepare the Benincasa hispida Cogniaux (Benincasae Semen) extract. 50 mg of the dried Benincasa hispida Cogniaux (Benincasae Semen) extract was dissolved in 1 ml of each extraction solvent, and solids insoluble in the solvent were removed by centrifugation, and only the supernatant was used as a sample.

### Example 4: Changes in Growth of Skin Microorganisms by Benincasa Hispida Cogniaux (Benincasae Semen) Extract

4-1. Microorganisms and Culture Media

Staphylococcus aureus (Hereinafter referred to as S. aureus) was used as the skin harmful bacteria, and Staphylococcus epidermidis (Hereinafter referred to as S. epidermidis) was used as the skin beneficial bacteria. S. aureus KCTC 1916 and S. epidermidis KCTC 1917 which were used in the experiment, were purchased from the Center for Biological Resources (Korean Collection for Type Cultures, Korea). The composition of the medium used for culturing the two microorganisms is shown in Table 2 below.

TABLE 2

| | | | |
|---|---|---|---|
| S. epidermidis | NB medium | Nutrient Broth | 0.8 g |
| KCTC 1917 | (100 ml) | (Agar) | 1.5 g |
| | | Water | Fill to a total |
| | | | volume of 100 ml |
| S. aureus | TSB medium | Tryptic Soy Broth | 3.0 g |
| KCTC 1916 | (100 ml) | (Agar) | 1.5 g |
| | | Water | Fill to a total |
| | | | volume of 100 ml |

4-2. Changes in Growth of Individual Skin Resident Microorganisms by Benincasa hispida Cogniaux (Benincasae Semen) Extract

S. aureus KCTC 1916 strain stock was spread on TSB (Tryptic Soy Broth, Becton Dickinson and Company, USA) agar plate and cultured at 37° C. for 24 hours. Then, one single colony was picked up and inoculated into 5 ml of TSB liquid medium, and pre-cultured overnight at 37° C. and 250 rpm. The pre-cultured S. aureus KCTC 1916 strain was inoculated into 20 ml of TSB liquid medium so that O.D.₆₀₀ became 0.05, and 200 μl (1% (v/v)) of Benincasa hispida Cogniaux (Benincasae Semen) extract extracted with methanol was added. In the control group, methanol was added at a concentration of 1% (v/v) instead of Benincasa hispida Cogniaux (Benincasae Semen) extract. Then, the growth degree was confirmed by measuring the absorbance at 600 nm using an Optizen 2120 UV plus spectrophotometer (Mecasys Co., Ltd., Daejeon, Korea) every 1 hour while culturing at 37° C. and 250 rpm.

Also, a stock of S. epidermidis KCTC 1917 was cultured in the same manner as above to measure the growth rate, except that the stock was cultured by spreading on an NB (nutrients broth, Becton Dickinson and Company) agar plate.

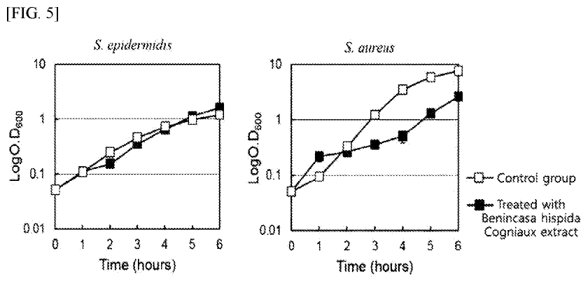

As a result of the measurement, as shown in FIG. 5, it could be confirmed that by the Benincasa hispida Cogniaux (Benincasae Semen) extract extracted with methanol, the growth rate of S. epidermidis was increased and the growth rate of S. aureus was decreased. Specifically, the doubling time of S. epidermidis was decreased by about 7.52% in the group treated with Benincasa hispida Cogniaux (Benincasae Semen) extract (71.41 minutes) compared to the control group (77.22 minutes).

On the other hand, the doubling time of S. aureus was increased by about 321.27% in the group treated with Benincasa hispida Cogniaux (Benincasae Semen) extract (147.95 minutes) compared to the control group (35.12 minutes).

4-3. Changes in Growth of Skin Resident Microorganisms by Benincasa hispida Cogniaux (Benincasae Semen) Extract Extracted with Methanol

Microorganisms were pre-cultured in the same manner as in Example 4-2, and the pre-cultured S. aureus KCTC 1916 strain and S. epidermidis KCTC 1917 strain were mixed and inoculated in 20 ml of NB liquid medium so that O.D.₆₀₀ became 0.05. 200 μl (1% (v/v)) of Benincasa hispida Cogniaux (Benincasae Semen) extract extracted with methanol was added, and in the case of control group, methanol was added at a concentration of 1% (v/v), followed by incubation at 37° C. and 250 rpm for 3 hours. Thereafter, 100 μl of the culture solution was spread on the NB agar plate, and cultured at 37° C. for 24 hours to confirm the colony forming unit (CFU).

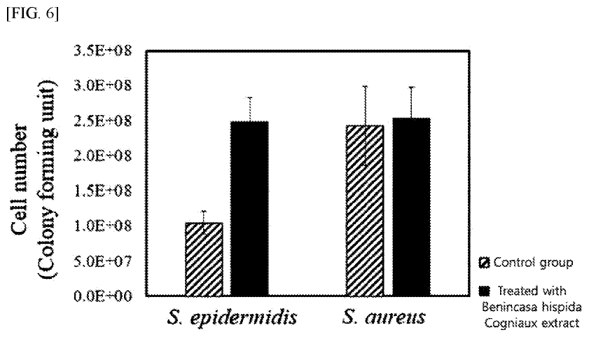

As a result, as shown in FIG. 6, it could be confirmed that by the treatment of the Benincasa hispida Cogniaux (Benincasae Semen) extract extracted with methanol, the growth of S. epidermidis was remarkably increased.

4-4. Changes in Growth of Skin Resident Microorganisms by Benincasa hispida Cogniaux (Benincasae Semen) Extract Extracted with 95% Ethanol

Except for the treatment using Benincasa hispida Cogniaux (Benincasae Semen) extract extracted with 95% ethanol at different concentrations (0 to 0.5 g/l), the growth degree of microorganisms was confirmed in the same manner as in 4-3 above.

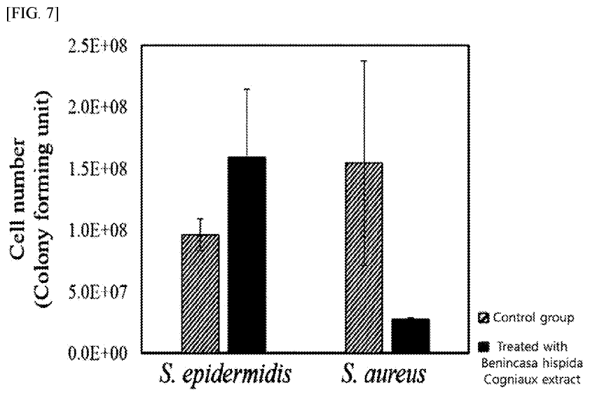

As a result, as shown in FIG. 7, it could be confirmed that by the treatment of the Benincasa hispida Cogniaux (Benincasae Semen) extract extracted with 95% ethanol, the growth of S. aureus was remarkably decreased and the growth of S. epidermidis was significantly increased.

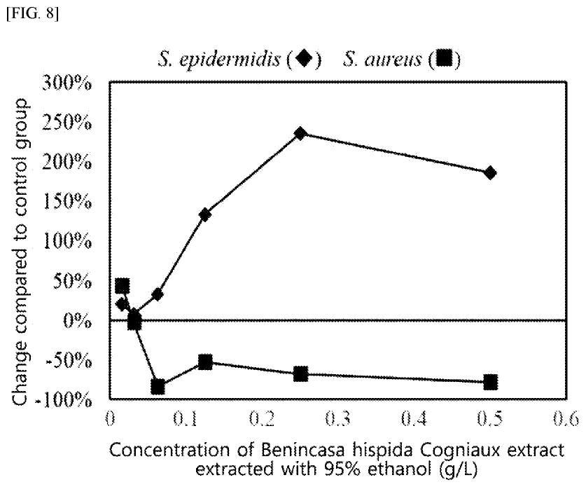

In addition, the degree of microbial growth was confirmed by the treatment of the Benincasa hispida Cogniaux (Benincasae Semen) extract extracted with 95% ethanol at different concentrations (0 to 0.5 g/l). As a result, as shown in FIG. 8, it could be confirmed that by the treatment of the Benincasa hispida Cogniaux (Benincasae Semen) extract extracted with 95% ethanol, the growth of S. epidermidis was increased and the growth of S. aureus was remarkably decreased, depending on the concentration.

### Example 5: Preparation of Dolichoris Semen Extract

To 5 g of Dolichoris Semen extract, 50 ml of 95% ethanol or methanol (medicament:solvent ratio of 1:10) was added, and the primary extract was obtained by shaking in a constant temperature water bath at 50° C. for 3 hours. The primary extract was filtered with a filter paper, and the filtered extract was concentrated and dried to prepare Dolichoris Semen extract. 50 mg of the dried Dolichoris Semen extract was dissolved in 1 ml of each extraction solvent, and solids insoluble in the solvent were removed by centrifugation, and only the supernatant was used as a sample.

### Example 6: Changes in Growth of Skin Microorganisms by Dolichoris Semen Extract

6-1. Microorganisms and Culture Media

Staphylococcus aureus (Hereinafter referred to as S. aureus) was used as the skin harmful bacteria, and Staphylococcus epidermidis (Hereinafter referred to as S. epidermidis) was used as the skin beneficial bacteria.

S. aureus KCTC 1916 and S. epidermidis KCTC 1917 which were used in the experiment, were purchased from the Center for Biological Resources (Korean Collection for Type Cultures, Korea). The composition of the medium used for culturing the two microorganisms is shown in Table 3 below.

TABLE 3

| | | | |
|---|---|---|---|
| S. epidermidis | NB medium | Nutrient Broth | 0.8 g |
| KCTC 1917 | (100 ml) | (Agar) | 1.5 g |
| | | Water | Fill to a total |
| | | | volume of 100 ml |
| S. aureus | TSB medium | Tryptic Soy Broth | 3.0 g |
| KCTC 1916 | (100 ml) | (Agar) | 1.5 g |
| | | Water | Fill to a total |
| | | | volume of 100 ml |

6-2. Changes in Growth of Individual Skin Resident Microorganisms by Dolichoris Semen Extract

S. aureus KCTC 1916 strain stock was spread on TSB (Tryptic Soy Broth, Becton Dickinson and Company, USA) agar plate and cultured at 37° C. for 24 hours.

Then, one single colony was picked up and inoculated into 5 ml of TSB medium, and pre-cultured overnight at 37° C. and 250 rpm. The pre-cultured S. aureus KCTC 1916 strain was inoculated into 20 ml of TSB liquid medium so that O.D.₆₀₀ became 0.05, and 200 μl (1% (v/v)) of Dolichoris Semen extract extracted with methanol was added. In the control group, methanol was added at a concentration of 1% (v/v) instead of the Dolichoris Semen extract. Then, the growth degree was confirmed by measuring the absorbance at 600 nm using an Optizen 2120 UV plus spectrophotometer (Mecasys Co., Ltd., Daejeon, Korea) every 1 hour while culturing at 37° C. and 250 rpm.

Also, a stock of S. epidermidis KCTC 1917 was cultured in the same manner as above to measure the growth rate, except that the stock was cultured by spreading on an NB (nutrients broth, Becton Dickinson and Company) agar plate.

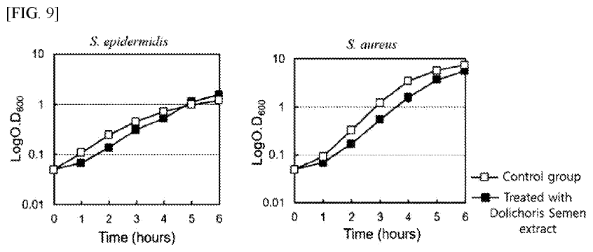

As a result of the measurement, as shown in FIG. 9, it could be confirmed that by the Dolichoris Semen extract extracted with methanol, the growth rate of S. epidermidis was increased and the growth rate of S. aureus was decreased. Specifically, the doubling time of S. epidermidis was decreased by about 21.2% in the group treated with the Dolichoris Semen extract (60.77 minutes) compared to the control group (77.22 minutes).

On the other hand, the doubling time of S. aureus was increased by about 13.58% in the group treated with the Dolichoris Semen extract (39.82 minutes) compared to the control group (35.12 minutes).

6-3. Changes in Growth of Skin Resident Microorganisms by Dolichoris Semen Extract Extracted with Methanol

Microorganisms were pre-cultured in the same manner as in Example 6-2, and the pre-cultured S. aureus KCTC 1916 strain and S. epidermidis KCTC 1917 strain were mixed and inoculated in 20 ml of NB liquid medium so that O.D.₆₀₀ became 0.05. 200 μl (1% (v/v)) of Dolichoris Semen extract extracted with methanol was added, and in the case of control group, methanol was added at a concentration of 1% (v/v), followed by incubation at 37° C. and 250 rpm for 3 hours. Thereafter, 100 μl of the culture solution was spread on the NB agar plate, and cultured at 37° C. for 24 hours to confirm the colony forming unit (CFU).

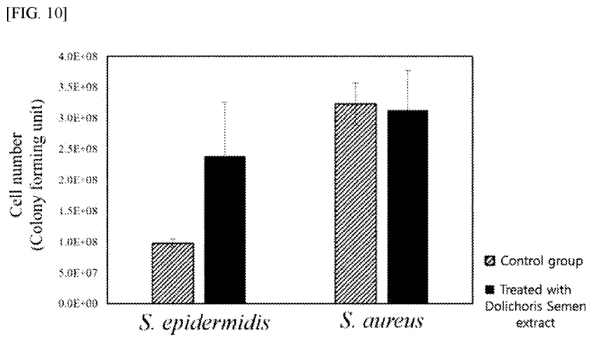

As a result, as shown in FIG. 10, it could be confirmed that by the treatment of the Dolichoris Semen extract extracted with methanol, the growth of S. epidermidis was remarkably increased.

6-4. Changes in Growth of Skin Resident Microorganisms by Dolichoris Semen Extract Extracted with 95% Ethanol

Except for the treatment using Dolichoris Semen extract extracted with 95% ethanol at different concentrations (0 to 0.5 g/l), the growth degree of microorganisms was confirmed in the same manner as in 6-3 above.

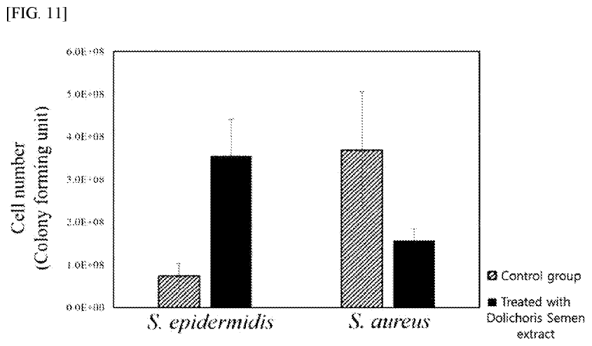

As a result, as shown in FIG. 11, it could be confirmed that by the treatment of the Dolichoris Semen extract extracted with 95% ethanol, the growth of S. aureus was remarkably decreased and the growth of S. epidermidis was significantly increased.

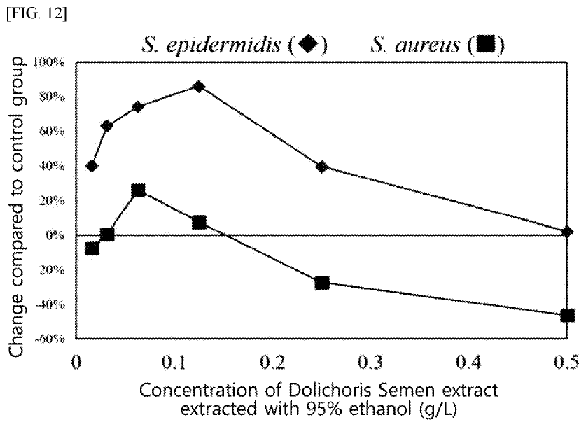

In addition, the degree of microbial growth was confirmed by the treatment of the Dolichoris Semen extract extracted with 95% ethanol at different concentrations (0 to 0.5 g/l). As a result, as shown in FIG. 12, it could be confirmed that by the treatment of Dolichoris Semen extract extracted with 95% ethanol, the growth of S. epidermidis was increased and the growth of S. aureus was remarkably decreased, depending on the concentration.

### Example 7: Preparation of Mori Folium Extract

To 5 g of Mori Folium, 50 ml of 95% ethanol or methanol (medicament:solvent ratio of 1:10) was added, and the primary extract was obtained by shaking in a constant temperature water bath at 50° C. for 3 hours. The primary extract was filtered with a filter paper, and the filtered extract was concentrated and dried to prepare Mori Folium extract. 50 mg of the dried Mori Folium extract was dissolved in 1 ml of each extraction solvent, and solids insoluble in the solvent were removed by centrifugation, and only the supernatant was used as a sample.

### Example 8: Changes in Growth of Skin Microorganisms by Mori Folium Extract

8-1. Microorganisms and Culture Media

Staphylococcus aureus (Hereinafter referred to as S. aureus) was used as the skin harmful bacteria, and Staphylococcus epidermidis (Hereinafter referred to as S. epidermidis) was used as the skin beneficial bacteria.

S. aureus KCTC 1916 and S. epidermidis KCTC 1917 which were used in the experiment, were purchased from the Center for Biological Resources (Korean Collection for Type Cultures, Korea). The composition of the medium used for culturing the two microorganisms is shown in Table 4 below.

TABLE 4

| | | | |
|---|---|---|---|
| S. epidermidis | NB medium | Nutrient Broth | 0.8 g |
| KCTC 1917 | (100 ml) | (Agar) | 1.5 g |
| | | Water | Fill to a total |
| | | | volume of 100 ml |
| S. aureus | TSB medium | Tryptic Soy Broth | 3.0 g |
| KCTC 1916 | (100 ml) | (Agar) | 1.5 g |
| | | Water | Fill to a total |
| | | | volume of 100 ml |

8-2. Changes in Growth of Individual Skin Resident Microorganisms by Mori Folium Extract

S. aureus KCTC 1916 strain stock was spread on TSB (Tryptic Soy Broth, Becton Dickinson and Company, USA) agar plate and cultured at 37° C. for 24 hours. Then, one single colony was picked up and inoculated into 5 ml of TSB medium, and pre-cultured overnight at 37° C. and 250 rpm. The pre-cultured S. aureus KCTC 1916 strain was inoculated into 20 ml of TSB liquid medium so that O.D.₆₀₀ became 0.05, and 200 μl (1% (v/v)) of Mori Folium extract extracted with methanol was added. In the control group, methanol was added at a concentration of 1% (v/v) instead of Mori Folium extract. Then, the growth degree was confirmed by measuring the absorbance at 600 nm using an Optizen 2120 UV plus spectrophotometer (Mecasys Co., Ltd., Daejeon, Korea) every 1 hour while culturing at 37° C. and 250 rpm.

Also, a stock of S. epidermidis KCTC 1917 was cultured in the same manner as above to measure the growth rate, except that the stock was cultured by spreading on an NB (nutrients broth, Becton Dickinson and Company) agar plate.

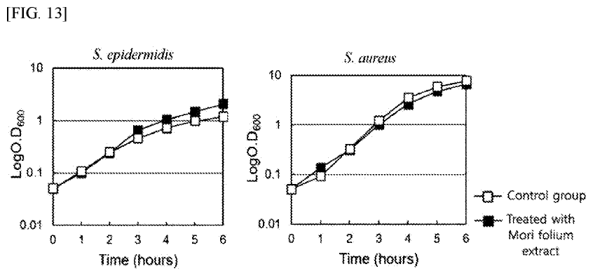

As a result of the measurement, as shown in FIG. 13, it could be confirmed that by Mori Folium extract extracted with methanol, the growth rate of S. epidermidis was increased and the growth rate of S. aureus was decreased. Specifically, the doubling time of S. epidermidis was decreased by about 42.45% in the group treated with Mori Folium extract (40.44 minutes) compared to the control group (77.22 minutes). On the other hand, the doubling time of S. aureus was increased by about 21.04% in the group treated with Mori Folium extract (42.51 minutes) compared to the control group (35.12 minutes).

8-3. Changes in Growth of Skin Resident Microorganisms by Mori Folium Extract Extracted with Methanol

Microorganisms were pre-cultured in the same manner as in Example 8-2, and the pre-cultured S. aureus KCTC 1916 strain and S. epidermidis KCTC 1917 strain were mixed and inoculated in 20 ml of NB liquid medium so that O.D.₆₀₀ became 0.05. 200 μl (1% (v/v)) of Mori Folium extract extracted with methanol was added, and in the case of control group, methanol was added at a concentration of 1% (v/v), followed by incubation at 37° C. and 250 rpm for 3 hours. Thereafter, 100 μl of the culture solution was spread on the NB agar plate, and cultured at 37° C. for 24 hours to confirm the colony forming unit (CFU).

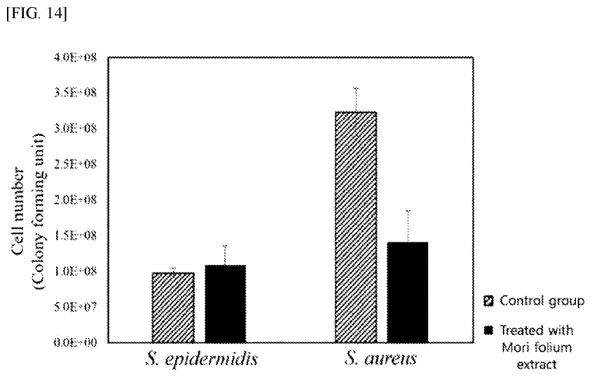

As a result, as shown in FIG. 14, it could be confirmed that by the treatment of the Mori Folium extract extracted with methanol, the growth of S. aureus was remarkably inhibited.

8-4. Changes in Growth of Skin Resident Microorganisms by Mori Folium Extract Extracted with 95% Ethanol

Except for the treatment using Mori Folium extract extracted with 95% ethanol at different concentrations (0 to 0.5 g/l), the growth degree of microorganisms was confirmed in the same manner as in 8-3 above.

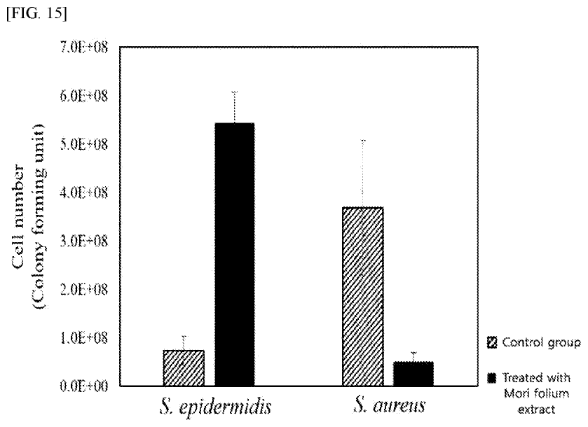

As a result, as shown in FIG. 15, it could be confirmed that by the treatment of the Mori Folium extract extracted with 95% ethanol, the growth of S. aureus was remarkably decreased and the growth of S. epidermidis was significantly increased.

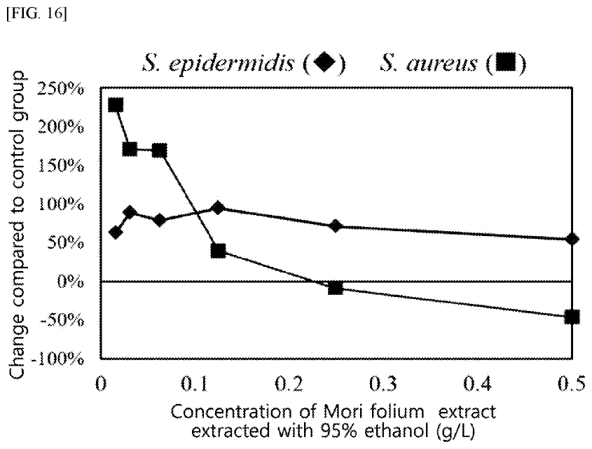

In addition, the degree of microbial growth was confirmed by the treatment of the Mori Folium extract extracted with 95% ethanol at different concentrations (0 to 0.5 g/l). As a result, as shown in FIG. 16, it could be confirmed that by the Mori Folium extract extracted with methanol, the growth of S. aureus was remarkably decreased.

### Example 9: Preparation of Coicis Semen Extract

To 5 g of Coicis Semen extract, 50 ml of 95% ethanol or methanol (medicament:solvent ratio of 1:10) was added, and the primary extract was obtained by shaking in a constant temperature water bath at 50° C. for 3 hours. The primary extract was filtered with a filter paper, and the filtered extract was concentrated and dried to prepare Coicis Semen extract. 50 mg of dried Coicis Semen extract was dissolved in 1 ml of each extraction solvent, and solids insoluble in the solvent were removed by centrifugation, and only the supernatant was used as a sample.

### Example 10: Changes in Growth of Skin Microorganisms by Coicis Semen Extract

10-1. Microorganisms and Culture Media

Staphylococcus aureus (Hereinafter referred to as S. aureus) was used as the skin harmful bacteria, and Staphylococcus epidermidis (Hereinafter referred to as S. epidermidis) was used as the skin beneficial bacteria.

S. aureus KCTC 1916 and S. epidermidis KCTC 1917 which were used in the experiment, were purchased from the Center for Biological Resources (Korean Collection for Type Cultures, Korea). The composition of the medium used for culturing the two microorganisms is shown in Table 5 below.

TABLE 5

| | | | |
|---|---|---|---|
| S. epidermidis | NB medium | Nutrient Broth | 0.8 g |
| KCTC 1917 | (100 ml) | (Agar) | 1.5 g |
| | | Water | Fill to a total |
| | | | volume of 100 ml |
| S. aureus | TSB medium | Tryptic Soy Broth | 3.0 g |
| KCTC 1916 | (100 ml) | (Agar) | 1.5 g |
| | | Water | Fill to a total |
| | | | volume of 100 ml |

10-2. Changes in Growth of Individual Skin Resident Microorganisms by Coicis Semen Extract

S. aureus KCTC 1916 strain stock was spread on TSB (Tryptic Soy Broth, Becton Dickinson and Company, USA) agar plate and cultured at 37° C. for 24 hours. Then, one single colony was picked up and inoculated into 5 ml of TSB medium, and pre-cultured overnight at 37° C. and 250 rpm. The pre-cultured S. aureus KCTC 1916 strain was inoculated into 20 ml of TSB liquid medium so that O.D.₆₀₀ became 0.05, and 200 μl (1% (v/v)) of Coicis Semen extract extracted with methanol was added. In the control group, methanol was added at a concentration of 1% (v/v) instead of the Coicis Semen extract. Then, the growth degree was confirmed by measuring the absorbance at 600 nm using an Optizen 2120 UV plus spectrophotometer (Mecasys Co., Ltd., Daejeon, Korea) every 1 hour while culturing at 37° C. and 250 rpm.

Also, a stock of S. epidermidis KCTC 1917 was cultured in the same manner as above to measure the growth rate, except that the stock was cultured by spreading on an NB (nutrients broth, Becton Dickinson and Company) agar plate.

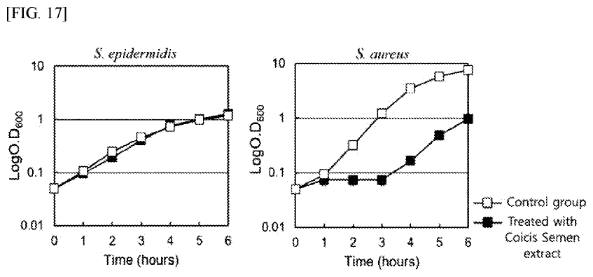

As a result of the measurement, as shown in FIG. 17, it could be confirmed that by the Coicis Semen extract extracted with methanol, the growth rate of S. epidermidis was increased and the growth rate of S. aureus was decreased. Specifically, the doubling time of S. epidermidis was decreased by about 22.13% in the group treated with the Coicis Semen extract (60.13 minutes) compared to the control group (77.22 minutes).

On the other hand, the doubling time of S. aureus was increased by about 25.77% in the group treated with the Coicis Semen extract (44.17 minutes) compared to the control group (35.12 minutes).

10-3. Changes in Growth of Skin Resident Microorganisms by Coicis Semen Extract Extracted with Methanol

Microorganisms were pre-cultured in the same manner as in Example 10-2, and the pre-cultured S. aureus KCTC 1916 strain and S. epidermidis KCTC 1917 strain were mixed and inoculated in 20 ml of NB liquid medium so that O.D.₆₀₀ became 0.05. 200 μl (1% (v/v)) of Coicis Semen extract extracted with methanol was added, and in the case of control group, methanol was added at a concentration of 1% (v/v), followed by incubation at 37° C. and 250 rpm for 3 hours. Thereafter, 100 μl of the culture solution was spread on the NB agar plate, and cultured at 37° C. for 24 hours to confirm the colony forming unit (CFU).

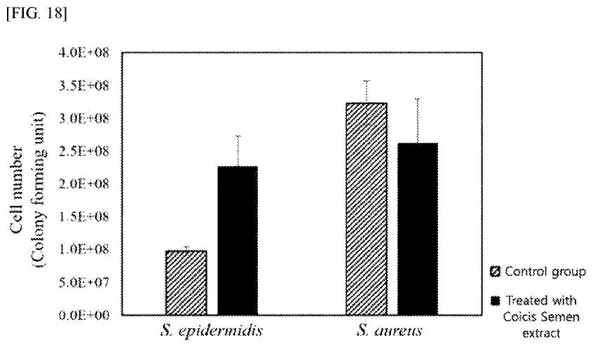

As a result of the measurement, as shown in FIG. 18, it could be confirmed that by the treatment of the Coicis Semen extract extracted with methanol, the growth of S. aureus was remarkably inhibited and the growth of S. epidermidis was remarkably increased.

10-4. Changes in Growth of Skin Resident Microorganisms by Coicis Semen Extract Extracted with 95% Ethanol

Except for the treatment using Coicis Semen extract extracted with 95% ethanol at different concentrations (0 to 0.5 g/l), the growth degree of microorganisms was confirmed in the same manner as in 10-3 above.

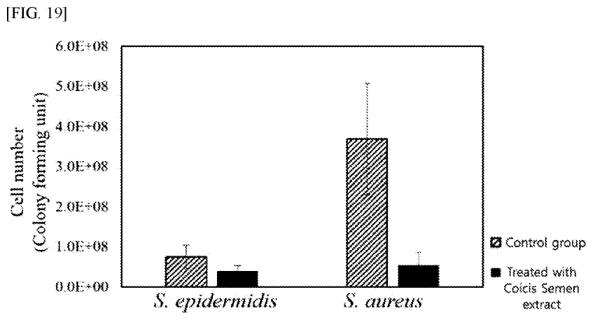

As a result, as shown in FIG. 19, it could be confirmed that by the treatment of the Coicis Semen extract extracted with 95% ethanol, the growth of S. aureus was remarkably decreased.

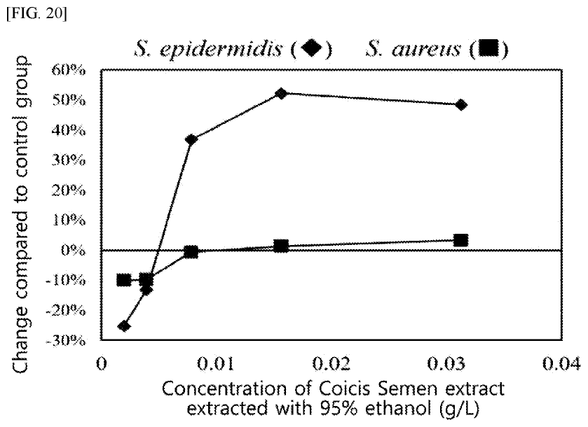

In addition, the degree of microbial growth was confirmed by the treatment of the Coicis Semen extract extracted with 95% ethanol at different concentrations (0 to 0.5 g/l). As a result, as shown in FIG. 20, it could be confirmed that by the Coicis Semen extract extracted with 95% ethanol, the growth of S. epidermidis was remarkably increased.

### Example 11: Preparation of Carthami Fructus Extract

To 5 g of Carthami Fructus, 50 ml of 95% ethanol or methanol (medicament:solvent ratio of 1:10) was added, and the primary extract was obtained by shaking in a constant temperature water bath at 50° C. for 3 hours. The primary extract was filtered with a filter paper, and the filtered extract was concentrated and dried to prepare Carthami Fructus extract. 50 mg of dried Carthami Fructus extract was dissolved in 1 ml of each extraction solvent, and solids insoluble in the solvent were removed by centrifugation, and only the supernatant was used as a sample.

### Example 12: Changes in Growth of Skin Microorganisms by Carthami Fructus Extract

12-1. Microorganisms and Culture Media

Staphylococcus aureus (Hereinafter referred to as S. aureus) was used as the skin harmful bacteria, and Staphylococcus epidermidis (Hereinafter referred to as S. epidermidis) was used as the skin beneficial bacteria.

S. aureus KCTC 1916 and S. epidermidis KCTC 1917 which were used in the experiment, were purchased from the Center for Biological Resources (Korean Collection for Type Cultures, Korea). The composition of the medium used for culturing the two microorganisms is shown in Table 6 below.

TABLE 6

| | | | |
|---|---|---|---|
| S. epidermidis | NB medium | Nutrient Broth | 0.8 g |
| KCTC 1917 | (100 ml) | (Agar) | 1.5 g |
| | | Water | Fill to a total |
| | | | volume of 100 ml |
| S. aureus | TSB medium | Tryptic Soy Broth | 3.0 g |
| KCTC 1916 | (100 ml) | (Agar) | 1.5 g |
| | | Water | Fill to a total |
| | | | volume of 100 ml |

12-2. Changes in Growth of Individual Skin Resident Microorganisms by Carthami Fructus Extract

S. aureus KCTC 1916 strain stock was spread on TSB (Tryptic Soy Broth, Becton Dickinson and Company, USA) agar plate and cultured at 37° C. for 24 hours. Then, one single colony was picked up and inoculated into 5 ml of TSB medium, and pre-cultured overnight at 37° C. and 250 rpm. The pre-cultured S. aureus KCTC 1916 strain was inoculated into 20 ml of TSB liquid medium so that O.D.₆₀₀ became 0.05, and 200 μl (1% (v/v)) of Carthami Fructus extract extracted with methanol was added. In the control group, methanol was added at a concentration of 1% (v/v) instead of the Carthami Fructus extract. Then, the growth degree was confirmed by measuring the absorbance at 600 nm using an Optizen 2120 UV plus spectrophotometer (Mecasys Co., Ltd., Daejeon, Korea) every 1 hour while culturing at 37° C. and 250 rpm.

Also, a stock of S. epidermidis KCTC 1917 was cultured in the same manner as above to measure the growth rate, except that the stock was cultured by spreading on an NB (nutrients broth, Becton Dickinson and Company) agar plate.

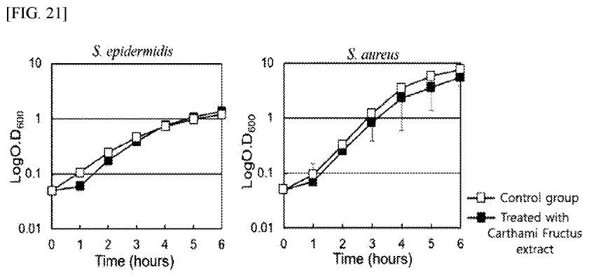

As a result of the measurement, as shown in FIG. 21, it could be confirmed that by the Carthami Fructus extract extracted with methanol, the growth rate of S. epidermidis was increased and the growth rate of S. aureus was decreased. Specifically, the doubling time of S. epidermidis was decreased by about 36.14% in the group treated with the Carthami Fructus extract (49.31 minutes) compared to the control group (77.22 minutes).

On the other hand, the doubling time of S. aureus was increased by about 1.28% in the group treated with the Carthami Fructus extract (35.57 minutes) compared to the control group (35.12 minutes).

12-3. Changes in Growth of Skin Resident Microorganisms by Carthami Fructus Extract Extracted with Methanol

Microorganisms were pre-cultured in the same manner as in Example 12-2, and the pre-cultured S. aureus KCTC 1916 strain and S. epidermidis KCTC 1917 strain were mixed and inoculated in 20 ml of NB liquid medium so that O.D.₆₀₀ became 0.05. 200 μl (1% (v/v)) of Carthami Fructus extract extracted with methanol was added, and in the case of control group, methanol was added at a concentration of 1% (v/v), followed by incubation at 37° C. and 250 rpm for 3 hours. Thereafter, 100 μl of the culture solution was spread on the NB agar plate, and cultured at 37° C. for 24 hours to confirm the colony forming unit (CFU).

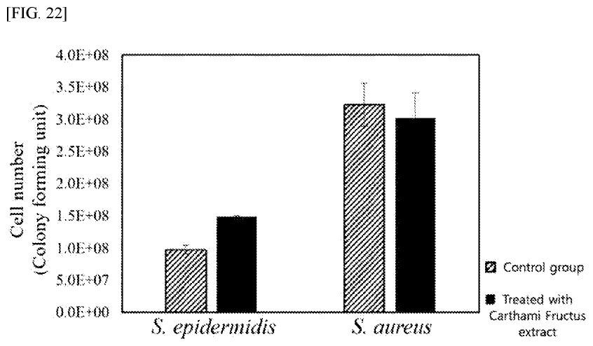

As a result of the measurement, as shown in FIG. 22, it could be confirmed that by treatment of the Carthami Fructus extract extracted with methanol, the growth of S. aureus was inhibited and the growth of S. epidermidis was increased.

12-4. Changes in Growth of Skin Resident Microorganisms by Carthami Fructus Extract Extracted with 95% Ethanol

Except for the treatment using Carthami Fructus extract extracted with 95% ethanol at different concentrations (0 to 0.5 g/l), the growth degree of microorganisms was confirmed in the same manner as in 12-3 above.

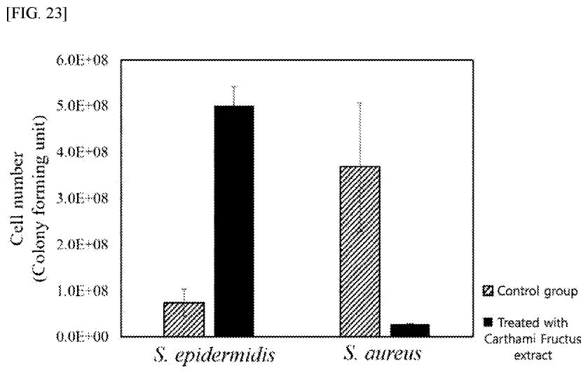

As a result, as shown in FIG. 23, it could be confirmed that by the treatment of the Carthami Fructus extract extracted with 95% ethanol, the growth of S. aureus was remarkably decreased, whereas the growth of S. epidermidis was significantly increased.

Those of ordinary skill in the art to which the present disclosure pertains will understand that the present disclosure can be implemented in a modified form without departing from the essential characteristics of the present disclosure. Therefore, the disclosed embodiments are to be considered in an illustrative rather than a restrictive sense. The scope of the present disclosure is indicated in the claims rather than the foregoing description, and all differences within the scope equivalent thereto should be construed as being included in the present disclosure.

1. A composition for improving microbial flora, comprising a herbal medicine extract as an active ingredient.

2. The composition of claim 1, wherein the herbal medicine is Persicae Semen, Benincasa hispida Cogniaux, Dolichoris Semen, Mori Folium, Coicis Semen, or Carthami Fructus.

3. The composition of claim 1, wherein the herbal medicine extract is extracted with one or more solvents selected from the group consisting of water, alcohol having 1 to 4 carbon atoms, ethyl acetate, diethyl ether, dichloromethane, and acetone.

4. The composition of claim 1, wherein the herbal medicine extract is included in an amount of 0.005 to 10% by weight based on the total weight of the composition.

5. The composition of claim 1, wherein the composition inhibits growth of harmful bacteria and promotes growth of beneficial bacteria.

6. The composition of claim 5, wherein the harmful bacteria is Staphylococcus aureus.

7. The composition of claim 5, wherein the beneficial bacteria is Staphylococcus epidermidis.

8. A cosmetic composition for improving skin microbial flora, comprising a herbal medicine extract as an active ingredient.

9. The cosmetic composition of claim 8, wherein the herbal medicine is Persicae Semen, Benincasa hispida Cogniaux, Dolichoris Semen, Mori Folium, Coicis Semen, or Carthami Fructus.

10. The cosmetic composition of claim 8, wherein the cosmetic composition is a formulation selected from the group consisting of gels, emulsions, suspensions, microemulsions, microcapsules, microgranules, ionic vesicle dispersants, non-ionic vesicle dispersants, creams, skin toners, lotions, powders, ointments, sprays, concealer sticks, aerosols, and mask packs.

11. A detergent composition, comprising a herbal medicine extract as an active ingredient.

12. The detergent composition of claim 11, wherein the herbal medicine is Persicae Semen, Benincasa hispida Cogniaux, Dolichoris Semen, Mori Folium, Coicis Semen, or Carthami Fructus.

13. The detergent composition of claim 11, wherein the detergent composition is selected from the group consisting of shampoos, rinses, hair conditioners, body washes, soaps, hand washes, laundry detergents, and kitchen detergents.
